(19) United States
(12) Patent Application Publication
(10) Pub. No.: US 2022/0361766 A1
(43) Pub. Date: Nov. 17, 2022

(54) MAGNETIC RESONANCE APPARATUS WITH A PATIENT POSITIONING APPARATUS

(71) Applicant: Siemens Healthcare GmbH, Erlangen (DE)

(72) Inventor: Stephan Biber, Erlangen (DE)

(21) Appl. No.: 17/739,320

(22) Filed: May 9, 2022

(30) Foreign Application Priority Data
May 11, 2021 (DE) 10 2021 204 738.6

(51) Int. Cl. A61B 5/055; A61B 5/00; A61G 13/02

(52) U.S. Cl. CPC A61B 5/055; A61B 5/704; A61G 13/02

(57) ABSTRACT

A magnetic resonance apparatus with a patient positioning apparatus is disclosed herein. The patient positioning apparatus includes a patient table for positioning a patient on an upper side of the patient table and a table positioning structure for movably positioning the patient table. The patient positioning apparatus is configured to position the patient by way of a movement of the patient table relative to the table positioning structure in the magnetic resonance apparatus, wherein the table positioning structure includes at least one spring element for sprung positioning of the patient table on the table positioning structure.

The present patent document claims the benefit of German Patent Application No. 10 2021 204 738.6, filed May 11, 2021, which is hereby incorporated by reference in its entirety.

### TECHNICAL FIELD

The disclosure relates to a magnetic resonance apparatus with a patient positioning apparatus.

### BACKGROUND

In medical technology, imaging by magnetic resonance (MR), also referred to as magnetic resonance tomography (MRT) or magnetic resonance imaging (MRI), is distinguished by high soft tissue contrast levels. Herein, a human or animal patient may be positioned in an examination space of a magnetic resonance apparatus. During a magnetic resonance scan, radio frequency (RF) transmit pulses may be radiated into the patient with the aid of a transmit coil unit of the magnetic resonance apparatus. Nuclear spins are excited in the patient by the transmit pulses generated, so that magnetic resonance signals are triggered. The magnetic resonance signals are received as medical image data by one or more receive coil units, in particular local coil units, of the magnetic resonance apparatus and are used for the reconstruction of magnetic resonance mappings. The receive coil unit may be a spine coil unit positioned under the patient.

During a magnetic resonance examination, the patient may be positioned on a patient table of a patient positioning apparatus. The spinal column coil unit may be integrated, in particular, into the patient positioning apparatus. In order to achieve a high signal-to-noise ratio in the magnetic resonance signals received by the spinal column coil unit, the spinal column coil unit may be positioned as closely as possible to the patient.

Very thin patient tables, in particular table boards, may be used. A possible reason therefor lies in bringing the patient as close as possible to the spinal column coil unit. However, under a high patient load, (e.g., 100 to 300 kg), thin patient tables may undergo severe bending. Thin but stiff table boards are complex to manufacture. One reason therefor is that carbon fibers or similar materials are not suitable for magnetic resonance apparatuses. This bending of the table board may be allowed for by an additional spacing between the patient table, in particular the table board and a table positioning structure lying thereunder, which carries the patient table, and which may include the spinal column coil unit. However, the additional spacing may lead to a poorer image quality, e.g., for the many cases in which the patient weighs less than 150 kg.

### SUMMARY AND DESCRIPTION

It may be regarded as an object of the present disclosure at least partially to overcome the disadvantages of the prior art described above.

The scope of the present disclosure is defined solely by the appended claims and is not affected to any degree by the statements within this summary. The present embodiments may obviate one or more of the drawbacks or limitations in the related art.

Accordingly, a magnetic resonance apparatus for capturing medical image data of a patient is proposed. The magnetic resonance apparatus includes a patient positioning apparatus, wherein the patient positioning apparatus includes a patient table for positioning a patient on an upper side of the patient table and a table positioning structure for movable positioning of the patient table. The table positioning structure may be arranged at least partially on the underside arranged opposite to the upper side of the patient table. The patient positioning apparatus is configured to position the patient by way of a movement of the patient table relative to the table positioning structure in the magnetic resonance apparatus. The table positioning structure therein includes at least one spring element for sprung positioning of the patient table on the table positioning structure.

Advantageously, the patient table is arranged sprung by the at least one spring element on the table positioning structure. Advantageously, by the spring mounting, any flexing and/or deformation of the patient table may be absorbed and/or compensated for. Advantageously, a mechanically more flexible pressing mechanism, which is independent of the table board deformation caused by the weight of the patient, may thus be provided. Any requirements placed on the rigidity of the patient table, in particular a table board of a patient table, may thereby advantageously be lessened.

The patient table may be configured, in particular, as a patient table board, or abbreviated: table board. The patient table or table board may be a movable part of the patient positioning apparatus. By contrast therewith, the table positioning structure is an immovable part of the patient positioning apparatus. The upper side of the patient table may be an upper side of the table board. The upper side of the patient table may have an extent that is large enough so that a human patient, in particular an adult patient, may be mounted lying thereupon. For example, the surface has a length in a range of 200 to 300 cm and a width in a range of 40 to 80 cm. The thickness of the patient table may be in a range of 1 to 20 cm.

The upper side of the patient table may be oriented horizontally during operation of the magnetic resonance apparatus, e.g., perpendicular to the direction of gravity.

The table positioning structure may be arranged beneath the patient table, e.g., offset in the direction of gravity. The patient table is arranged movably on the table positioning structure. The patient table may be moved, (e.g., displaced), with the aid of an electric motor on the table positioning structure. The magnetic resonance apparatus may include a control unit for moving the patient table on the table positioning structure.

The magnetic resonance apparatus may include a bore. The bore may be circular or elliptical. The bore may have a central axis which may be defined, for example, as the z-axis. The patient table may be moved, (e.g., displaced), on the table positioning structure along the z-axis.

Advantageously, the movement of the patient table on the table positioning structure is suitable for positioning the patient in the magnetic resonance apparatus. For example, the patient may be moved into an examination space of the magnetic resonance apparatus. In particular, the magnetic resonance apparatus may have an isocenter about which the magnetic resonance apparatus has a particularly homogeneous magnetic field so that magnetic resonance signals may be recorded there in particularly high quality. The isocenter of the magnetic resonance apparatus may be the midpoint of the volume of homogeneity of the magnetic field. Advantageously, a region of the patient from which image data is to be captured may be brought, by movement of the patient table relative to the table positioning structure, into the isocenter.

A further embodiment of the magnetic resonance apparatus provides that the patient table includes at least one roller for moving the patient table relative to the table positioning structure. The at least one roller may include a ball roller, a cylindrical roller, a wheel, and/or a disk. During a movement of the patient table, the patient table may roll on the at least one roller relative to the table positioning structure. Advantageously, a rolling movement has only a slight mechanical resistance so that the movement of the patient table relative to the table positioning structure may be carried out particularly easily.

A further embodiment of the magnetic resonance apparatus provides that the at least one spring element includes at least one mechanically compressible material and/or at least one spring. The at least one mechanically compressible material may include at least one foam material. The at least one spring may include at least one compression spring.

A further embodiment of the magnetic resonance apparatus provides that the at least one spring element include MR-compatible material. For example, the MR compatible material may include plastics and/or non-magnetic metal, in particular steel. In particular, the material has a low proton density and/or the protons of the material have a short T2 time of less than 100 microseconds. Advantageously, thereby the at least one spring element is less visible in any magnetic resonance mapping.

A further embodiment of the magnetic resonance apparatus provides that the table positioning structure includes a spinal column coil unit, wherein an upper side of the spinal column coil unit close to the patient is arranged between the at least one spring element and the patient table. The at least one spring element may be arranged, for example, between the spinal column coil unit as a whole and the table positioning structure. The at least one spring element may be arranged, for example, between a portion of the spinal column coil unit and the table positioning structure. In particular, the at least one spring element may also be integrated wholly or partially into the spinal column coil unit.

In a load-free state of the patient table, the underside of the patient table, (that is, the side opposite to the upper side of the patient table), may have a spacing of less than 30 mm from the spinal column coil unit. A load-free state may exist, for example, if no patient is lying on the patient table.

The magnetic resonance apparatus may have a main magnetic field with a magnetic field strength of not more than 1.5 tesla, e.g., less than 1 tesla. At low magnetic field strengths, a small spacing from the spinal column coil unit is particularly advantageous. The reason is that the signal-to-noise ratio falls off more strongly with increasing separation between the spinal column coil unit, in particular any receive antenna in the spinal column coil unit, and the patient, in particular the scanning region in the patient, than in systems with a higher magnetic field strength.

Advantageously, the at least one spring element absorbs any force acting upon the spinal column coil unit which acts, for example, by way of the patient table on the spinal column coil unit. If, for example, the patient table is deformed by a patient load, in the regions of the patient table at which a particularly strong deformation occurs, a particularly large force may act upon the spinal column coil unit so that spring elements situated in these regions are particularly strongly compressed. This is advantageous, in particular, if in a load-free state, which is in particular, without any loading by the patient, the patient table has only a small spacing from the spinal column coil unit, for example, less than 30 mm, or even touches the spinal column coil unit.

The spring deflection of the at least one spring element may be selected so that it is great enough to be able to absorb normally occurring deformations of the patient table. Any deformations of the patient table may thus advantageously be compensated for.

The spring deflection of the at least one spring element may be selected so that it is great enough to be able to absorb normally occurring deformations of the patient table.

The spinal column coil unit may be configured, in particular, rigid so that given a pressure acting in the context of a magnetic resonance examination, it does not deform at all or deforms only slightly.

The at least one spring element may be arranged, in particular, between a non-yielding, in particular rigid, portion of the table positioning structure and the spinal column coil unit. The non-yielding portion of the table positioning structure may include a structure arranged on an inner surface of the bore and/or the inner surface of the bore itself. The inner surface of the bore may be connected, in particular structurally, to a body coil of the magnetic resonance apparatus.

A further embodiment of the magnetic resonance apparatus provides that the table positioning structure, in particular the spinal column coil unit and/or the patient table, includes at least one roller for moving the patient table relative to the table positioning structure. The patient table may roll on the at least one roller. The at least one roller may include a ball roller, a cylindrical roller, a wheel, or a disk. During a movement of the patient table, the patient table may roll on the at least one roller relative to the table positioning structure. Advantageously, a rolling movement has only a slight mechanical resistance so that the movement of the patient table relative to the table positioning structure may be carried out particularly easily.

The table positioning structure may include a spinal column coil unit, wherein the table positioning structure, in particular the spinal column coil unit and/or the patient table, include a plurality of rollers for moving the patient table relative to the table positioning structure, wherein a first portion of the plurality of rollers is arranged on the spinal column coil unit and a further part of the plurality of rollers is arranged beside the spinal column coil unit.

Advantageously, at least part of the weight force may be jointly carried by rollers arranged on the spinal column coil unit. For example, a portion of the load of the patient table is absorbed via rollers arranged laterally on the patient table (and conducted, for example, into a body coil of the magnetic resonance apparatus); the remaining load that may arise, in particular, due to a bending of the patient table onto the spinal column coil unit may be absorbed via rollers arranged on the spinal column coil unit.

A further embodiment of the magnetic resonance apparatus provides that the table positioning structure and/or the patient table includes a total of 3 to 12 rollers in the region of the spinal column coil unit for moving the patient table relative to the table positioning structure. Such a number is particularly suitable for achieving a free movement and/or an even load distribution of the patient table. In particular, any deformation of the patient table may thereby be “absorbed” at a plurality of points and “transferred” to the spinal column coil unit.

A further embodiment of the magnetic resonance apparatus provides that the patient positioning apparatus is configured so that the movement of the patient table relative to the table positioning structure takes place in a movement direction that is oriented parallel to the upper side of the patient table, wherein the spinal column coil unit has a midline that is oriented parallel to the movement direction, at least one of the rollers being arranged on the midline and/or the rollers being arranged symmetrically to the midline and/or being at least partially arranged at the ends of the spinal column coil unit in relation to the movement direction.

For example, the midline of the spinal column coil unit is parallel to and/or coincident with the central axis of the bore and/or the z-axis of the magnetic resonance apparatus.

Advantageously, by way of a symmetrical arrangement of the rollers, an even load distribution may be achieved. By way of an arrangement at the ends, advantageously a conduction of the load over a particularly large region, in particular along the z-axis, may be achieved.

A further embodiment of the magnetic resonance apparatus provides that the spinal column coil unit includes a plurality of sub-units which may be tilted relative to one another.

In particular, for patient tables with a very flexible table board, the spinal column coil unit may be configured to adapt to the shape of the table board (e.g., dependent upon the patient load). For example, a bending mechanism may be provided, in particular in the center of the spinal column coil unit which allows the spinal column coil unit to bend by, for example, 0-10°. It is also conceivable that the spinal column coil unit is itself subdivided into a plurality of individual sub-units which are movable relative to one another on the surface. The sub-units may move largely independently of one another and conform themselves to the patient table and/or the patient.

A further embodiment of the magnetic resonance apparatus provides that the spinal column coil unit has a housing which has a thinner wall thickness on the patient side than in other regions of the housing.

At least one antenna, (e.g., a receive antenna), may be arranged within the housing of the spinal column coil unit. In particular, magnetic resonance signals may be received with the at least one antenna.

Advantageously, by different wall thicknesses, it may be achieved that as small a spacing as possible exists between the upper side of the patient table and the at least one antenna, but that the spinal column coil unit nevertheless has a high degree of mechanical stability. For example, the housing has a wall thickness on an upper side of the patient side in a range of 2 to 4 mm, whereas the other housing parts have a wall thickness in a range of 4 to 10 mm.

A further embodiment of the magnetic resonance apparatus provides that the movable positioning of the patient table on the table positioning structure is constructed so that the patient table may be moved from a first region in which the patient table is not positioned on the spinal column coil unit, into a second region in which the patient table is positioned at least partially on the spinal column coil unit.

The spinal column coil unit and/or the patient table may include a beveled and/or rounded edge so that on a movement of the patient table from the first region to the second region, the spinal column coil unit and the patient table slide over one another by the beveled and/or rounded edge.

For example, a head-side edge of the patient table, in particular of the table board, may be beveled in order to prevent a collision when moving in over the spinal column coil unit and to create a soft transition.

Further advantages, characteristics, and details of the disclosure become apparent from the description below of exemplary embodiments and from the drawings. Parts which correspond to one another are provided with the same reference signs in all the drawings.

### BRIEF DESCRIPTION OF THE DRAWINGS

FIG. 1 is a schematic representation of an example of a magnetic resonance apparatus.

FIG. 2 is a cross-sectional view of an example of a bore of a magnetic resonance apparatus patient table on which rollers are fixed.

FIG. 3 is a cross-sectional view of an example of a bore of a magnetic resonance apparatus spinal column coil unit on which rollers are fixed.

FIG. 4 is a plan view of an example of an arrangement a patient table and a table positioning structure.

FIG. 5 is a side view of an example of an arrangement of a patient table and a table positioning structure in different states.

FIG. 6 is an example of a spinal column coil unit which is bendable in the middle.

FIG. 7 is an example of a spinal column coil unit with three sub-units which may be tilted relative to one another.

### DETAILED DESCRIPTION

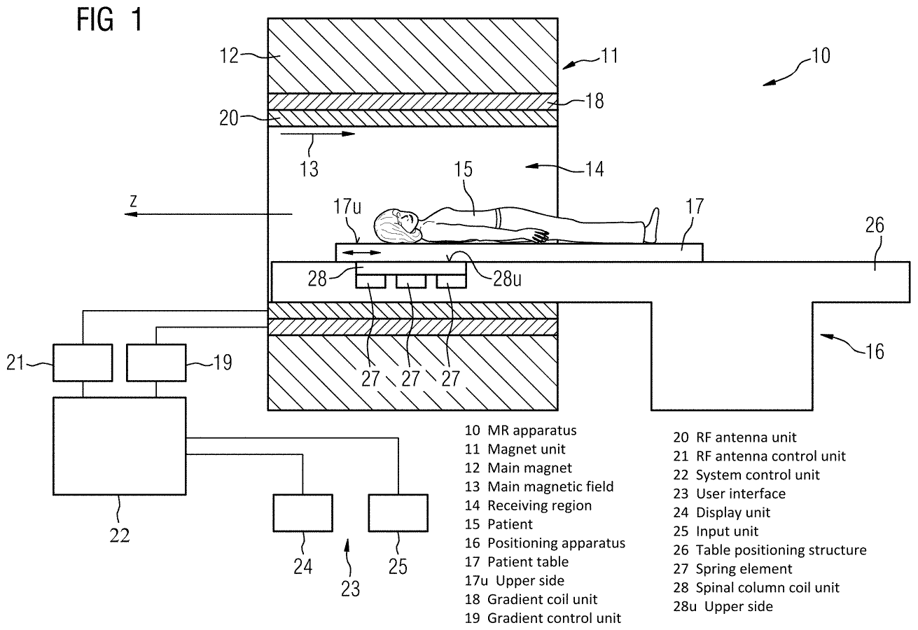

FIG. 1 shows a schematic view of a magnetic resonance apparatus 10. The magnetic resonance apparatus 10 includes a magnet unit 11 which has a main magnet 12 for generating a strong and, particularly, temporally constant main magnetic field 13. Advantageously, the magnetic field strength of the main magnetic field 13 is less than 1 tesla. In addition, the magnetic resonance apparatus 10 includes a patient receiving region 14 for accommodating a patient 15. In the present exemplary embodiment, the patient receiving region 14 is configured cylindrical in the form of a bore and is surrounded cylindrically in a peripheral direction about a z-axis by the magnet unit 11. In principle, however, an embodiment of the patient receiving region 14 deviating therefrom is readily conceivable.

The patient 15 may be pushed by a patient positioning apparatus 16 of the magnetic resonance apparatus 10 into the patient receiving region 14. For this purpose, the patient positioning apparatus 16 has a patient table 17 which is configured to be movable within the patient receiving region 14, on the upper side 28u of which patient table the patient 15 is positioned, and a table positioning structure 26 for movable mounting of the patient table 17. In particular, the patient table 17 may be moved on the table positioning structure 26 parallel to the z-axis. In order to move the patient table 17, the magnetic resonance apparatus 10 may include an operating console (not shown in detail here) which may be arranged directly beside the bore in the magnetic resonance apparatus 10. With the aid of the operating console, a motor may be controlled, by which the patient table 17 may be moved back and forth on the table positioning structure 26.

The magnet unit 11 also has a gradient coil unit 18 for generating magnetic field gradients that are used for position encoding during an imaging process. The gradient coil unit 18 is controlled by a gradient control unit 19 of the magnetic resonance apparatus 10. The magnet unit 11 further includes a radio frequency antenna unit 20 configured as a body coil firmly integrated into the magnetic resonance apparatus 10. The radio frequency antenna unit 20 is controlled by a radio frequency antenna control unit 21 of the magnetic resonance apparatus 10 and radiates radio frequency magnetic resonance sequences into an examination space which is substantially formed by a patient accommodating region 14 of the magnetic resonance apparatus 10. By this mechanism, an excitation of atomic nuclei by the main magnetic field 13 generated by the main magnet 12 takes place. Through relaxation of the excited atomic nuclei, medical image data are generated in the form of magnetic resonance signals.

In order to be able to receive the magnetic resonance signals with a high signal-to-noise ratio, local coil units may be used because the local coils may be mounted close to the body of the patient 15. In the present example, the magnetic resonance apparatus 10 includes a spinal column coil unit 28 positioned under the patient 15 as part of a table positioning structure. Therein, the patient table 17 is mounted in the region of the spinal column coil unit 28 on the spinal column coil unit 28. The spinal column coil unit 28 is itself mounted on spring elements 27 of the table positioning structure 26 so that the patient table 17 is also mounted on the spring elements 27 of the table positioning structure 26. In particular, the upper side 28u of the spinal column coil unit close to the patient is arranged between the spring elements 27 and the patient table 17. The spring effect of the spring elements 27 is therein transferred via the spinal column coil unit 28 to the patient table 17.

The spring elements 27 may include at least one mechanically compressible material, (e.g., at least one foam material), and/or at least one spring, (e.g., at least one compression spring). The spring elements 27 may include MR-compatible material, e.g., plastics and/or non-magnetic metal. In particular, the material has a low proton density and/or the protons of the material have a short T2 time of less than 100 microseconds. Advantageously, the spring elements 27 are thereby less visible in the reconstructed magnetic resonance mapping.

For controlling the main magnet 12, the gradient control unit 19 and, for controlling the radio frequency antenna control unit 21, the magnetic resonance apparatus 10 has a system control unit 22. The system control unit 22 centrally controls the magnetic resonance apparatus 10, for example, the execution of a pre-determined imaging gradient echo sequence. In addition, the system control unit 22 includes an evaluation unit (not shown in detail) for evaluating the magnetic resonance signals which are captured during the magnetic resonance examination. Furthermore, the magnetic resonance apparatus 10 includes a user interface 23 connected to the system control unit 22. Control information such as imaging parameters and reconstructed magnetic resonance mappings may be displayed on a display unit 24, (e.g., on at least one monitor), of the user interface 23 for medical operating personnel. In addition, the user interface 23 has an input unit 25 by which the information and/or parameters may be input by the operating medical personnel during a scanning procedure.

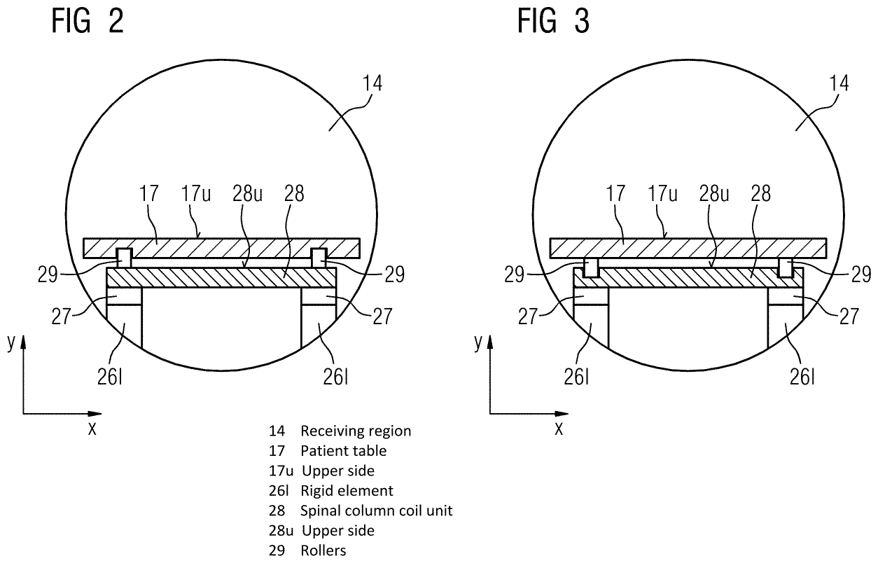

FIG. 2 shows a cross-section through the bore or the patient receiving region 14 of the magnetic resonance apparatus 10. The table positioning structure 26 herein has on each side a, particularly rigid, element 261, on each of which elements a spring element 27 is mounted. The spinal column coil unit 28 is mounted thereon. The patient table 17 is mounted on the spinal column coil unit 28. A roller 29 is arranged on the patient table 17, on each side thereof, for moving the patient table 17 relative to the table positioning structure 26. The rollers 29 are therein set into the patient table 17. Advantageously, the spacing of the upper edge of the roller from the upper side 28u of the spinal column coil unit 28 defines the spacing between the patient table 17 and the spinal column coil unit 28.

In FIG. 3, at least one roller 29 is arranged on both sides of the table positioning structure 26, in particular on the spinal column coil unit 28, for moving the patient table 17 relative to the table positioning structure 26. The rollers 29 are therein set into the spinal column coil unit 28. Advantageously, the spacing of the lower edge of the roller from the lower side of the patient table 17 defines the spacing between the patient table 17 and the spinal column coil unit 28.

It is also conceivable that both the table positioning structure 26, in particular the spinal column coil unit 28, and the patient table 17 each include one or more rollers 29. Advantageously, the table positioning structure 25 and/or the patient table 17 include a total of 3 to 12 rollers 29 in the region of the spinal column coil unit 28 for moving the patient table 17 relative to the table positioning structure 26.

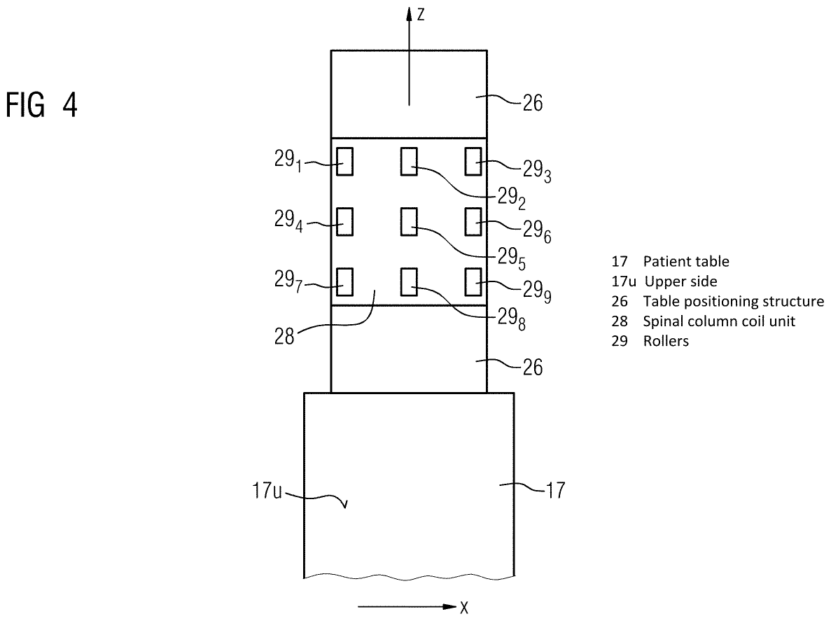

For example, the spinal column coil unit 28 shown in FIG. 4 includes at least three rows of 3 rollers each, that is a total of 9 rollers 29₁, 29₂, 29₃, 29₄, 29₅, 29₆, 29₇, 29₈, 29₉ in the region of the spinal column coil unit 28. By these rollers 29₁, 29₂, 29₃, 29₄, 29₅, 29₆, 29₇, 29₈, 29₉, the patient table may be moved, in particular displaced, in particular in the direction of the z-axis, which is in the z-direction.

The z-direction is oriented parallel to the upper side 17u of the patient table 17. A midline which is oriented parallel to the z-direction may be associated with the spinal column coil unit 28. The three rollers 29₂, 29₅, 29₈, are therein arranged on the midline. The three rollers 29₃, 29₆, 29₉ are arranged on the right side and the three rollers 29₁, 29₄, 29₇ are arranged at the left symmetrically to the midline. Furthermore, three rollers 29₁, 29₂, 29₃ are arranged in the z-direction on the upper end of the spinal column coil unit and three rollers 29₇, 29₈, 29₉ are arranged on the lower end of the spinal column coil unit.

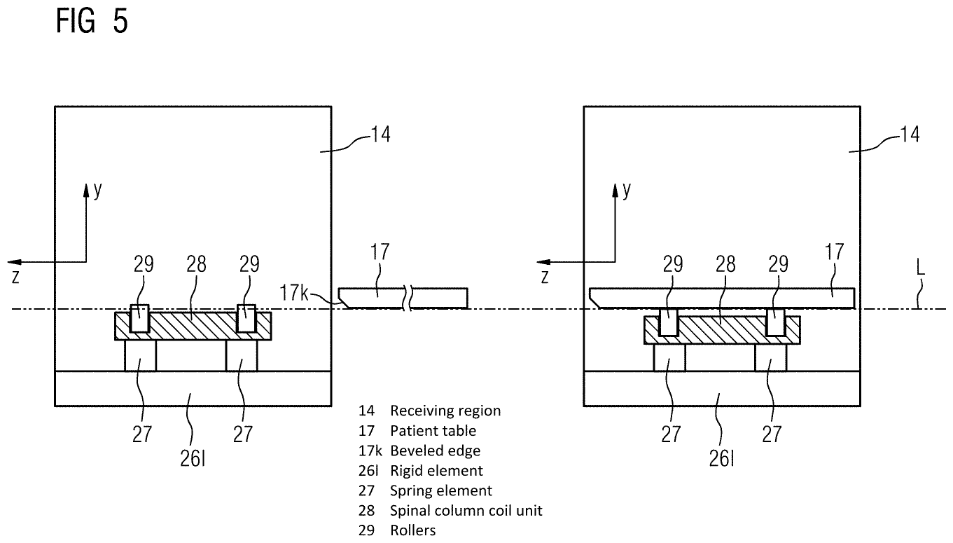

FIG. 5 shows a state, at left, in which the patient table 17 is still situated outside the bore. The patient table is arranged here in a first region in which the patient table 17 is not mounted on the spinal column coil unit 28. At right, a state is depicted in which the patient table 17 is situated inside the bore. The patient table is situated here in a second region in which the patient table 17 is not mounted on the spinal column coil unit 28. The patient table 17 may be moved along the z-direction from the first region into the second region.

Herein, the patient table 17 includes a beveled edge 17k so that on a movement of the patient table 17 from the first region into the second region, the spinal column coil unit 28, in particular the rollers 29 of the spinal column coil unit 28, and the patient table 17 slide over one another by the beveled edge 17k. The underside of the edge 17k (or the underside of the patient table) is situated during the whole of the movement process at a height L. If the edge 17k touches the roller 29, the latter is pushed downwardly by the incline of the edge 17k, and the spinal column coil unit deflects downwardly. The spring elements 27 are thereby compressed accordingly. In this way, a mechanical flexibility is advantageously provided which creates an optimum spacing between the upper side 17u of the patient table 17 (or the patient 15 lying thereon) and the spinal column coil unit 28.

Alternatively, or additionally, the spinal column coil unit 29 may also include a beveled edge in order to enable and/or support a collision-free sliding over one another.

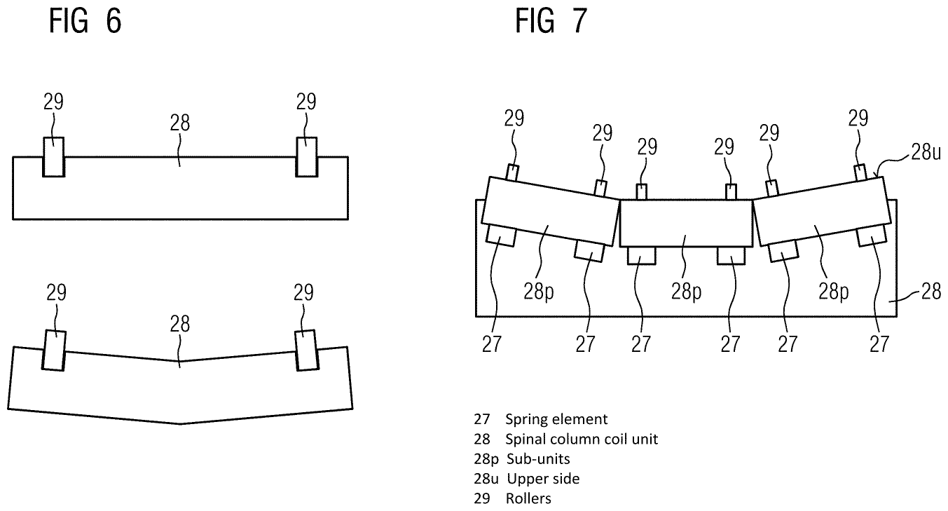

In particular for patient tables with a particularly flexible table board, it may be advantageous if the spinal column coil unit 28 may adapt to the shape of the table board, in particular dependent upon the patient load. Advantageously, this may be enabled by way of sub-units which may be tilted relative to one another. FIGS. 6 and 7 show two different embodiments of the spinal column coil unit 28 with a plurality of sub-units which may be tilted relative to one another. Shown in FIG. 6 at top is a spinal column coil unit 28 in a non-tilted state. At bottom, it is apparent that the spinal column coil unit 28 is configured to tilt a sub-unit (shown at left) of the spinal column coil unit 28 opposite a sub-unit (shown at right) of the spinal column coil unit 28. Thereby, a simple bending mechanism may advantageously be provided in the center which additionally allows the coil to be bent by, for example, 0-10°.

FIG. 7 shows a spinal column coil unit 28 which has a plurality of sub-units 28p movable relative to one another on the upper side 28u. The sub-units 28p may advantageously move largely independently of one another and so form themselves optimally to the patient table and/or the patient. The spring elements 29 are here integrated into the spinal column coil unit 28. The spinal column coil unit 28 may have a lower portion that is rigidly constructed. In an upper portion, the sub-units 28p that are movable relative to one another are arranged. The spring elements 29 are arranged in a central portion, which is between the lower and the upper portion.

The magnetic resonance apparatus described above in detail merely involves exemplary embodiments which may be modified by a person skilled in the art in a wide variety of ways without departing from the scope of the disclosure. Furthermore, the use of the indefinite article “a” or “an” does not preclude the possibility that the relevant features may also be present plurally. Similarly, the expression “unit” does not preclude the relevant components include a plurality of cooperating sub-components which may also be spatially distributed if relevant.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A magnetic resonance apparatus for capturing medical image data of a patient, the magnetic resonance apparatus comprising:
a patient positioning apparatus having:
a patient table for positioning the patient on an upper side of the patient table; and
a table positioning structure for movably positioning the patient table,
wherein the patient positioning apparatus is configured to position the patient by way of a movement of the patient table relative to the table positioning structure in the magnetic resonance apparatus,
wherein the table positioning structure comprises at least one spring element for sprung positioning of the patient table on the table positioning structure.

2. The magnetic resonance apparatus of claim 1, wherein the patient table comprises at least one roller for moving the patient table relative to the table positioning structure.

3. The magnetic resonance apparatus of claim 1, wherein the at least one spring element comprises at least one mechanically compressible material and/or at least one spring.

4. The magnetic resonance apparatus of claim 3, wherein the at least one mechanically compressible material comprises at least one foam material.

5. The magnetic resonance apparatus of claim 3, wherein the at least one spring comprises at least one compression spring.

6. The magnetic resonance apparatus of claim 1, wherein the at least one spring element includes MR-compatible material.

7. The magnetic resonance apparatus of claim 6, wherein the MR-compatible material comprises a plastic, a non-magnetic metal, or a combination thereof.

8. The magnetic resonance apparatus of claim 1, wherein the table positioning structure comprises at least one roller for moving the patient table relative to the table positioning structure.

9. The magnetic resonance apparatus of claim 1, wherein the table positioning structure comprises a spine coil, and
wherein an upper side of the spine coil close to the patient is arranged between the at least one spring element and the patient table.

10. The magnetic resonance apparatus of claim 9, wherein the spine coil and/or the patient table comprise at least one roller for moving the patient table relative to the table positioning structure.

11. The magnetic resonance apparatus of claim 9, wherein the table positioning structure and/or the patient table comprise a total of 3 to 12 rollers in a region of the spine coil for moving the patient table relative to the table positioning structure.

12. The magnetic resonance apparatus of claim 9, wherein the patient positioning apparatus is configured so that the movement of the patient table relative to the table positioning structure takes place in a movement direction oriented parallel to the upper side of the patient table,
wherein the spine coil has a midline which is oriented parallel to the movement direction,
wherein at least one roller is arranged on the midline and/or the at least one roller is arranged symmetrically to the midline and/or are at least partially arranged at ends of the spine coil in relation to the movement direction.

13. The magnetic resonance apparatus of claim 9, wherein the spine coil comprises a plurality of sub-units configured to be tilted relative to one another.

14. The magnetic resonance apparatus of claim 9, wherein the spine coil has a housing which has a wall thickness that is thinner on a side adjacent to the patient in comparison with other regions of the housing.

15. The magnetic resonance apparatus of claim 9, wherein the patient table on the table positioning structure is configured to be moved from a first region in which the patient table is not positioned on the spine coil into a second region in which the patient table is positioned at least partially on the spine coil.

16. The magnetic resonance apparatus of claim 15, wherein the spine coil and/or the patient table comprise a beveled edge and/or a rounded edge so that on the movement of the patient table from the first region into the second region, the spine coil and the patient table slide over one another by the beveled edge and/or the rounded edge.

17. The magnetic resonance apparatus of claim 1, wherein the magnetic resonance apparatus comprises a main magnetic field with a magnetic field strength of not more than 1.5 tesla.
